(19) United States
(12) Patent Application Publication
(10) Pub. No.: US 2009/0054830 A1
(43) Pub. Date: Feb. 26, 2009

(54) ELECTRONIC DEVICE FOR CONTROLLING THE INFUSION OF MEDICAL-GRADE CARBON DIOXIDE GAS

(76) Inventor: Paraguassu De Sa Roriz, Sao Paulo (BR)

Correspondence Address: SCHMEISER, OLSEN & WATTS, 22 CENTURY HILL DRIVE, SUITE 302, LATHAM, NY, 12110, US

(21) Appl. No.: 12/278,816

(22) PCT Filed: Sep. 27, 2006
(86) PCT No.: PCT/BR06/00200

§ 371 Date: Aug. 8, 2008

(30) Foreign Application Priority Data
Feb. 8, 2006 (BR) MU 8600192-2

(51) Int. Cl. A61M 37/00

(52) U.S. Cl. 604/23

(57) ABSTRACT

The present invention relates to an electronic device for controlling the infusion of medical-grade carbon device gas, comprising a gas infusion-control arrangement, formed by a supporting box.

### TECHNICAL FIELD

The present invention relates to an electronic equipment for controlling the infusion of medical-grade carbon dioxide gas, which is provided with an innovative conception and important technological and functional improvements, based on the most modern engineering concepts, and in accordance with the required norms and specifications, resulting in proper and functional characteristics, endowed with fundamental requirements of novelty and inventive step, thus leading to a number of real and extraordinary technical, practical and economical advantages.

### BACKGROUND OF THE INVENTION

The medical-grade carbonic gas, that is, carbon dioxide gas, is a non-toxic gas, and usually it is present as an intermediate of the cellular metabolism. This gas is known to be used for the treatment of several pathologies, such as arteriopathies, phlebopathies, vascular ulcers, psoriasis, and for the treatment of circulatory problems. However, carbon dioxide is also used in aesthetic applications, for example, for the treatment of cellulite, cutaneous flaccidity, local body fat, stretch marks, and others.

The difference between its use for aesthetic applications in relation to other medical treatments consists in the fact that the amount/dosage of the medical-grade carbonic gas is significantly inferior for aesthetic applications.

The technique consists of injecting the medical-grade carbonic gas into the subcutaneous cellular tissue located under the dermis, which is the deepest layer of the skin. This method aims to improve the local circulation and tissue oxygenation, as well as to produce stimulus for the formation of collagen, whose effect leads to a remarkable improvement of the appearance of cellulite and flaccidity, by means of the improvement of the perfusion and the tissue metabolism. In addition, it is a fast, comfortable and effective treatment for the great majority of patients.

For the appropriate application of this treatment, the physician needs to control speed, volume and time of the carbon dioxide gas infusion into the patient. The lack of precision in this operation can compromise the success of the treatment, as well as cause damages to the patient.

As it is known in the state of the art, there are currently available on the market several devices for infusion of medical-grade carbon dioxide gas, whereas the great majority of these devices is not specific for the use in aesthetic treatments, and they are used for other purposes, such as, for example, treatments with dilatation of coronary arteries for cardiovascular examinations.

The gas infusion devices known in the state of the art do not provide appropriate control of speed, volume and time for applications in the treatment of cellulite, flaccidity, among others; mainly, because, for these specific cases of aesthetic treatments, the pressure and volume requirements of the flow of carbonic gas are inferior than those other more-known medical treatments.

Another inconvenient characteristic that these products known in the state of the art present is related to the ergonomics of the device, whereas, for aesthetic treatments, applications made in the patient's body require a constant handling of the device by the physician.

Moreover, said products known in the state of the art necessitate electronic and digital mechanisms, which allow more precise controls and measurements of the carbon dioxide gas volume and flow applied into the patient.

Along the time, many studies have been carried out aiming to improve this constructive arrangement of devices for controlling the infusion of medical-grade carbon dioxide gas, attempting to minimize such inconveniences. As a result, it was developed the constructive arrangement described in the present invention, which made possible the conception based on an innovative device that presents a flow-control arrangement by means of an electronic device that analyzes, in real time, the gas flow to be delivered to the patient, in such a way to improve and optimize the application of the medical-grade carbon dioxide gas in treatments of cellulite, flaccidity, among others.

### SUMMARY OF THE INVENTION

The device for controlling the infusion of medical-grade carbon dioxide gas of the present invention consists of a box-shaped structure, which supports the entire arrangement, wherein said box has an ergonomic shape, and allows a better and more practical handling of the device. The control arrangement is disposed internally to said box, wherein said arrangement is composed mainly of input and output couplings, polymeric hoses, a flow valve, a shutdown valve and an electronic device for the analysis of the gas flow.

The flow valve is responsible for regulating the gas flow from the external cylinder of medical-grade carbon dioxide gas. The flow valve receives said gas at a flow rate of about 15 liters/minute, and with a constant pressure of about 2.5 atm; then said flow valve converts this flow into a flow rate from 5 to 100 ml/minute. The shutdown valve is responsible for opening and closing of the gas flow by means of polymeric hoses, and thus controls the application of said gas into the patient. The input coupling is connected to an external gas cylinder, while the output coupling is fitted into an infusion device appropriated for the treatment of desired applications.

After passed through the flow valve and through the shutdown valve, carbon dioxide gas is lead to an electronic device called “volumetric gas displacement measurer”, which detects the flow variations by means of a sensor, and by the detected current volume, said sensor sends information to a microprocessor that interprets it and then sends such values in digital form to the screen of a display. The display shows information about the current flow in real-time. Moreover, said microprocessor also calculates automatically the total volume in relation to the flow speed and in relation to the application time, and sends this information to the display. These real-time digital information helps and guides the physician, so that the professional can perform his/her work in a more precise way than methods and means known in the state of the art, wherein any variation in the flow regulating valve is instantly interpreted by the electronic device and sent to the digital display of said device for a precise reading.

One of the advantages of this new constructive arrangement is that the physician can have automated programming of infusion time or total amount to be delivered to the patient. With this electronic arrangement in action, the program evaluates the delivered total gas amount, and when the total amount previously programmed by the physician is reached, a sonorous signal is emitted, while an advice is exhibited at the device's display; and in the same way, it occurs when the device is programmed by infusion time.

The electronic device of the present invention also allows evaluating an excessive flow, alerting the physician to the need for flow reduction, as well as evaluates the flow that falls below appropriate standards for the treatment.

Moreover, the electronic device of the present invention also allows analyzing the total volume of delivered gas, as well as the application time, in order to determine the relationship between the use during a determined period of time, such as per day, per week, per month or per year.

It is therefore an objective of the present invention to provide an electronic device for controlling the infusion of medical-grade carbon dioxide gas, having regard to the proposed function, and with quality and safety, using a new constructive arrangement based on the gas flow control, by means of an electronic arrangement added to a valve arrangement.

### BRIEF DESCRIPTION OF THE DRAWINGS

In order to provide a clear visualization of the electronic device for controlling the infusion of medical-grade carbon dioxide gas of the present invention, reference may be had to the accompanying drawings for a better understanding of the following detailed description, wherein:

FIG. 1 is an external perspective view of the electronic device for controlling the infusion of medical-grade carbon dioxide gas of the present invention;

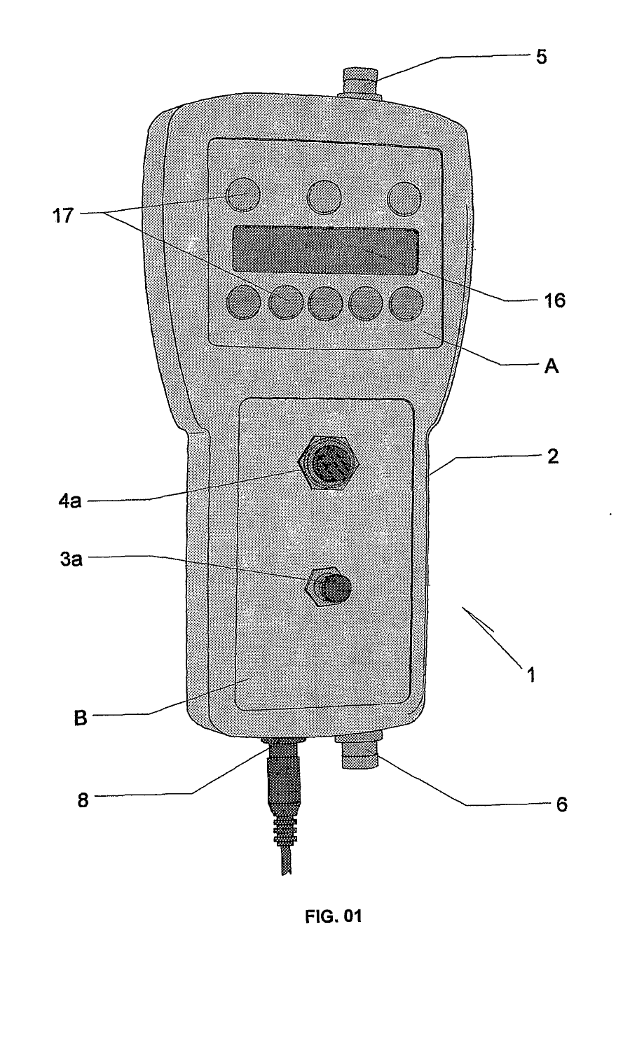

FIG. 2 is an internal top view of the electronic device for controlling the infusion of medical-grade carbon dioxide gas of the present invention.

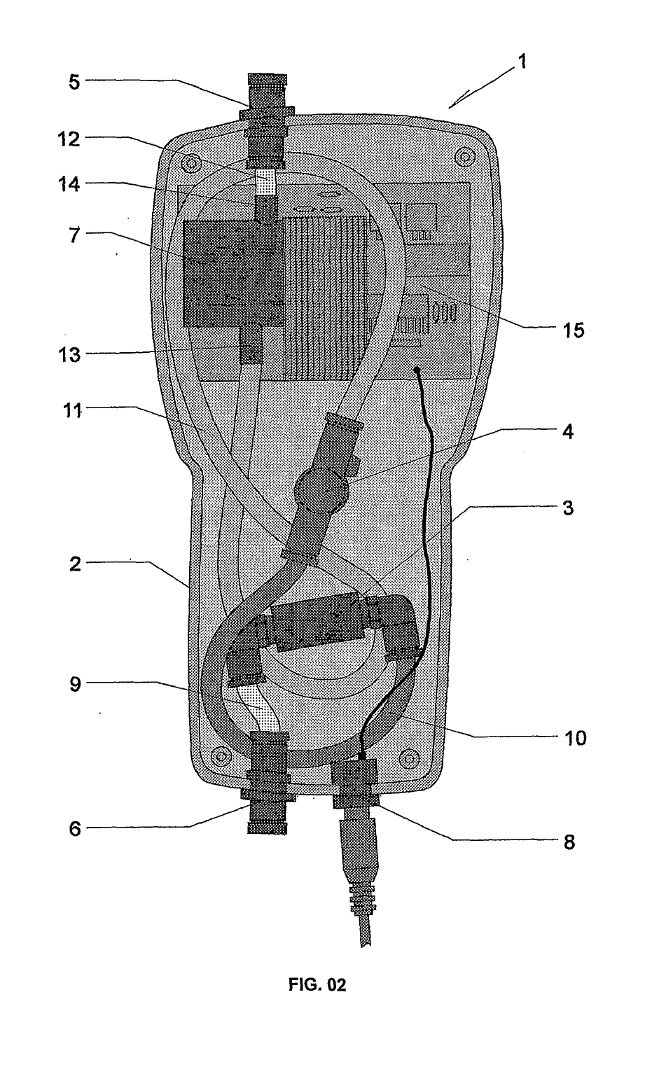

### DETAILED DESCRIPTION OF THE INVENTION

In accordance with these illustrations and their respective details, the electronic device for controlling the infusion of medical-grade carbon dioxide gas of the present invention comprises a gas infusion-control arrangement (1) formed by a supporting box (2), which has a main structural body, wherein said main structural body has a predominantly rectangular straight prism shape, with one of its half-portions having a width slightly greater than the width of the other half-portion. Said greater width half-portion (A) corresponds to the electronic control area, while the lesser width half-portion (B) corresponds to the carbon dioxide gas flow-control area.

The flow-control valve arrangement comprises a flow valve (3) and a shutdown valve (4). Each one of these valves comprises a handling body (3a) and (4a) displayed on the external surface of the supporting box (2).

The gas output coupling (5) is located on the superior portion of the supporting box (2), and the gas input coupling (6) is located on the inferior portion of the supporting box (2). The gas input coupling (6) is connected to an external cylinder of carbon dioxide gas, which is commercialized independently from the device of the present invention, and the gas output coupling (5) is connected to an infusion device, which is used by the physician for applications of the desired treatment. The device of the present invention can be connected directly to the electric power source, or a battery can be used to provide power to the electronic arrangement. In the case of electric power, a connector (8) for electric connection is located on the inferior portion of the supporting box (2).

In the internal portion of the supporting box (2) is located a arrangement composed of four polymeric hoses (9), (10), (11) and (12), which support appropriate pressure conditions, wherein the polymeric hose (9) connects the gas input coupling (6) to the flow valve (3); the polymeric hose (10) connects the flow valve (3) to the shutdown valve (4); the polymeric hose (11) connects the shutdown valve (4) to the sensor input (13) of the electronic flow measurer (15); and the polymeric hose (12) connects the sensor output (14) of the electronic flow measurer (15) to the gas outlet coupling (5).

The electronic flow measurer (15) is located internally to the supporting box (2) and includes a display panel (16) with handling buttons (17) on the external surface of the supporting box (2). Said electronic flow measurer (15) is provided with a sensor (7) comprising an input (13) and an output (14) from where the carbon dioxide gas flows. By means of said sensor (7), information about the gas flow in the arrangement is obtained, and the obtained data are sent to a microprocessor of the electronic flow measurer (15), which processes these data and prints them legibly to the physician on the panel display (16).

Moreover, the electronic flow measurer (15) is provided with a sonorous device that, together with the panel display, allows sending signals and warnings to the physician during the application. The electronic flow measurer (15) allows more control of the carbon dioxide gas flow, in a more practical way than conventional means. By means of said electronic arrangement, it is possible to preprogram the device in relation to the total amount of gas to be delivered, the application time, the excessive current flow, the inadequate current flow, among others. Moreover, for being an electronic device, it also allows calculating the total volume of gas delivered during a determined period of time, such as per day, per week, per month or per year.

In this way, when it is desired to carry out the process of subcutaneous infusion of carbon dioxide gas into a patient, the physician carries out the gas flow control by opening the shutdown valve (4) and regulating the flow valve (3), while the electronic flow measurer (15), in operation together with the panel display and its respective handling buttons (17), performs the automated calculation control of flow and infusion time of the gas delivered to the patient, thus becoming a more practical and precise treatment.

There is no knowledge of another constructive arrangement introduced in an electronic device for controlling the carbon dioxide gas infusion that encompasses all constructive and functional characteristics above-mentioned, and that is or was (directly or indirectly) as effective as the constructive arrangement disclosed in the present invention.

What is claimed is:

1. “ELECTRONIC DEVICE FOR CONTROLLING THE INFUSION OF MEDICAL-GRADE CARBON DIOXIDE GAS” comprising a gas infusion-control arrangement (1) formed by a supporting box (2), which has a main structural body, wherein said main structural body has a predominantly rectangular straight prism shape, with one of its half-portions having a width slightly greater than the width of the other half-portion; said greater width half-portion (A) corresponds to the electronic control area, while the lesser width half-portion (B) corresponds to the carbon dioxide gas flow-control area; a flow-control valve arrangement comprising a flow valve (3) and a shutdown valve (4), wherein each one of said valves comprises a handling body (3a) and (4a) displayed on the external surface of the supporting box (2); a gas output coupling (5) located on the superior portion of the supporting box (2) and a gas input coupling (6) located on the inferior portion of the supporting box (2); wherein said device can be connected directly to the electric power source, or a battery can be used to provide power to the electronic arrangement, and, in the case of electric power, a connector (8) for electric connection is located on the inferior portion of the supporting box (2); in the internal portion of the supporting box (2) is located a arrangement composed of four polymeric hoses (9), (10), (11) and (12), which support appropriate pressure conditions, wherein the polymeric hose (9) connects the gas input coupling (6) to the flow valve (3); the polymeric hose (10) connects the flow valve (3) to the shutdown valve (4); the polymeric hose (11) connects the shutdown valve (4) to the sensor input (13) of the electronic flow measurer (15); and the polymeric hose (12) connects the sensor output (14) of the electronic flow measurer (15) to the gas outlet coupling (5); the electronic flow measurer (15) is located internally to the supporting box (2) and includes a display panel (16) with handling buttons (17) on the external surface of the supporting box (2); said electronic flow measurer (15) is provided with a sensor (7) comprising an input (13) and an output (14) from where the carbon dioxide gas flows; and also, the electronic flow measurer (15) is provided with a sonorous device.
